(19) United States
(12) Patent Application Publication
(10) Pub. No.: US 2017/0095418 A1
(43) Pub. Date: Apr. 6, 2017

(54) NANO-DEVICES HAVING IMPELLERS FOR CAPTURE AND RELEASE OF MOLECULES

(71) Applicant: The Regents of the University of California, Oakland, CA (US)

(72) Inventors: Jeffrey I. Zink, Sherman Oaks, CA (US); Fuyuhiko Tamanoi, Los Angeles, CA (US); Eunshil Choi, Los Angeles, CA (US); Sarah A. Angelos, Corvallis, OR (US); Sanaz Kabehie, Los Angeles, CA (US); Andre E. Nel, Sherman Oaks, CA (US); Jie Lu, Los Angeles, CA (US)

(21) Appl. No.: 15/288,322

(22) Filed: Oct. 7, 2016

Related U.S. Application Data
(63) Continuation of application No. 12/812,359, filed on Jul. 9, 2010, now abandoned.
(60) Provisional application No. 61/006,597, filed on Jan. 23, 2008.

(51) Int. Cl. A61K 9/00; F04D 29/02; A61N 5/06; A61K 9/51; B01L 3/00

(52) U.S. Cl. CPC A61K 9/0097; A61K 9/5115; B01L 3/50273; A61N 5/062; F04D 29/026

(57) ABSTRACT

A nanodevice has a containment vessel defining a storage chamber therein and defining at least one port to provide transfer of molecules to or from the storage chamber, and a plurality of impellers attached to the containment vessel. The plurality of impellers are of a structure and are arranged to substantially block molecules from entering and exiting the storage chamber of the containment vessel when the impellers are static and are operable to impart motion to the molecules to cause the molecules to at least one of enter into or exit from the storage chamber of the containment vessel.

### CROSS-REFERENCE TO RELATED APPLICATION

This application claims priority to U.S. Provisional Application No. 61/006,597 filed Jan. 23, 2008, the entire contents of which are hereby incorporated by reference.

The U.S. Government has a paid-up license in this invention and the right in limited circumstances to require the patent owner to license others on reasonable terms as provided for by the terms of Grant Nos. CHE 0507929 and DMR 0346601, awarded by the National Science Foundation, and of Grant No. 32737, awarded by NIH.

### BACKGROUND

1. Field of Invention

The current invention relates to nano-devices, and more specifically to nano-devices having impellers for the capture and/or release of molecules.

2. Discussion of Related Art

Control of molecular transport in, through, and out of mesopores has important potential applications in nanoscience including fluidics and drug delivery. Surfactant-templated silica (Kresge, C. T.; Leonowicz, M. E.; Roth, W. J.; Vartuli, J. C.; Beck, J. S. Nature 1992, 359, 710-712) is a versatile material in which ordered arrays of mesopores can be easily synthesized, providing a convenient platform for attaching molecules that undergo large amplitude motions to control transport. Mesostructured silica is transparent (for photocontrol and spectroscopic monitoring), and can be fabricated into useful morphologies (thin films (Lu, Y. F.; Ganguli, R.; Drewien, C. A.; Anderson, M. T.; Brinker, C. J.; Gong, W. L.; Guo, Y. X.; Soyez, H.; Dunn, B.; Huang, M. H.; Zink, J. I. Nature 1997, 389, 364-368), particles (Kresge, C. T.; Leonowicz, M. E.; Roth, W. J.; Vartuli, J. C.; Beck, J. S. Nature 1992, 359, 710-712; Huh, S.; Wiench, J. W.; Yoo, J. C.; Pruski, M.; Lin, V. S. Y. Chem. Mater. 2003, 15, 4247-4256)) with designed pore sizes and structures. One method of controlling transport uses the photo-induced cis-trans isomerization of N═N bonds in azobenzene derivatives tethered to the interiors of mesopores. To date, the understanding of the light-responsive behavior of azobenzene-modified materials has been based on a static mechanism, where the effective pore sizes are varied by azobenzene existing in the trans or cis conformation.

Mesostructured inorganic materials functionalized with azobenzene (Liu, N. G.; Chen, Z.; Dunphy, D. R.; Jiang, Y. B.; Assink, R. A.; Brinker, C. J. Angew. Chem. Int. Ed. 2003, 42, 1731-1734; Liu, N. G.; Yu, K.; Smarsly, B.; Dunphy, D. R.; Jiang, Y. B.; Brinker, C. J. J. Am. Chem. Soc. 2002, 124, 14540-14541; Liu, N. G.; Dunphy, D. R.; Atanassov, P.; Bunge, S. D.; Chen, Z.; Lopez, G. P.; Boyle, T. J.; Brinker, C. J. Nano Lett. 2004, 4, 551-554; Alvaro, M.; Benitez, M.; Das, D.; Garcia, H.; Peris, E. Chem. Mater. 2005, 17, 4958-4964; Besson, E.; Mehdi, A.; Lerner, D. A.; Reye, C.; Corriu, R. J. P. J. Mater. Chem. 2005, 15, 803-809; Weh, K.; Noack, M.; Hoffmann, K.; Schroder, K. P.; Caro, J. Microporous Mesoporous Mater. 2002, 54, 15-26) have received significant attention owing to the photoactive responses of these hybrids, including control of the d-spacing of mesostructured materials (Liu, N. G.; Yu, K.; Smarsly, B.; Dunphy, D. R.; Jiang, Y. B.; Brinker, C. J. J. Am. Chem. Soc. 2002, 124, 14540-14541). Zeolitic membranes containing azobenzene were reported to exhibit photoswitchable gas permeation properties resulting from the trans-cis isomerization of azobenzene (Weh, K.; Noack, M.; Hoffmann, K.; Schroder, K. P.; Caro, J. Microporous Mesoporous Mater. 2002, 54, 15-26). Mesostructured silicates synthesized with azobenzene-bridged pores exhibit light-responsive changes in adsorption ability correlating with the dimensional changes of azobenzene that occur upon photoisomerization (Alvaro, M.; Benitez, M.; Das, D.; Garcia, H.; Peris, E. Chem. Mater. 2005, 17, 4958-4964). Additionally, the transport rate of ferrocene derivatives through an azobenzene-modified cubic-structured silica film to an electrode was photoresponsively controlled by changing the effective pore size (Liu, N. G.; Dunphy, D. R.; Atanassov, P.; Bunge, S. D.; Chen, Z.; Lopez, G. P.; Boyle, T. J.; Brinker, C. J. Nano Lett. 2004, 4, 551-554).

Although there has been substantial research activity in this field, there still remains a need for nano-devices that can selectively impel molecules into and out of a containment vessel and that can also keep the molecules substantially contained within the containment vessel when not being selectively impelled. There further remains a need for such nano-structures that can be useful for biological and biomedical applications.

### SUMMARY

A nanodevice according to some embodiments of the current invention has a containment vessel defining a storage chamber therein and defining at least one port to provide transfer of molecules to or from the storage chamber, and an impeller attached to the containment vessel. The impeller is operable to impart motion to the molecules to cause the molecules to at least one of enter into or exit from the storage chamber of the containment vessel, and the nanodevice has a maximum dimension of less than about 400 nm and greater than about 50 nm.

A nanodevice according to some embodiments of the current invention has a containment vessel defining a storage chamber therein and defining at least one port to provide transfer of molecules to or from the storage chamber, and a plurality of impellers attached to the containment vessel. The plurality of impellers are of a structure and are arranged to substantially block molecules from entering and exiting the storage chamber of the containment vessel when the impellers are static and are operable to impart motion to the molecules to cause the molecules to at least one of enter into or exit from the storage chamber of the containment vessel.

A composition of matter according to some embodiments of the current invention has a plurality of nanoparticles, each defining a storage chamber therein, and a guest material contained within the storage chambers defined by the nanoparticles, the guest material being substantially chemically non-reactive with the nanoparticles. The plurality of nanoparticles are operable to cause the guest material contained within the storage chambers to be ejected upon a transfer of energy to the plurality of nanoparticles from a source of energy external to the plurality of nanoparticles, and each nanoparticle of the plurality of nanoparticles has a maximum dimension of less than about 400 nm and greater than about 50 nm.

A method of administering at least one of a biologically active substance, a therapeutic substance, a neutraceutical substance, a cosmetic substance or a diagnostic substance according to some embodiments of the current invention includes administering a composition to at least one of a person, an animal, a plant, or an organism, the composition comprising nanoparticles therein, wherein the nanoparticles contain the at least one of a biologically active substance or a diagnostic substance therein; and illuminating the nanoparticles of the administered composition with light to cause the at least one of the biologically active substance or the diagnostic substance to be expelled from the nanoparticles.

### BRIEF DESCRIPTION OF THE DRAWINGS

Further objectives and advantages will become apparent from a consideration of the description, drawings, and examples.

FIG. 1 is a schematic illustration of a nanodevice according to an embodiment of the current invention. Pore interiors of light-activated mesostructured silica nanoparticles (LAMS) are functionalized with azobenzene derivatives. Continuous illumination at 413 nm causes a constant trans-cis photoisomerization about the N═N bond causing dynamic wagging motion of the azobenzene derivatives and results in the release of the molecules through and out of the mesopores.

FIGS. 2A and 2B are schematic illustrations of photoresponsive nanodevices functionalized with azobenzene derivatives according to two embodiments of the current invention. In FIG. 2A materials prepared by the co-condensation method (CCM) are derivatized with AzoH. In FIG. 2B materials prepared by the post-synthesis modification method (PSMM) are derivatized with AzoG1. For each system, the moveable phenyl ring of the azobenzene machine is illustrated by [symbol] , the tethered phenyl ring of the azobenzene machine by [symbol] , and the impelled molecule by [symbol] .

FIG. 3 is a schematic illustration of a nanodevice according to some embodiments of the current invention.

FIG. 4 shows an SEM image of silica nanoparticles and an illustration of the 2D hexagonal mesostructure according to an embodiment of the current invention. (The 2 nm diameter pores are not drawn to scale.)

FIGS. 5a-5c show plots of the luminescence intensity of Coumarin 540A at 540 nm in solution as a function of time measured at 1 sec intervals. The arrows indicate when the azobenzene excitation light (457 nm) is turned on. Release profile of Coumarin 540A from (FIG. 5a) AzoH-modified particles prepared by the CCM; (FIGS. 5b,5c) AzoG1 modified particles prepared by the PSMM. The profile of FIG. 5c demonstrates the on-off response to 457 nm excitation. Shaded regions indicate periods of time at which the azobenzene excitation light is on.

FIGS. 6A and 6B show characterization of the surfactant-extracted LAMS particles using scanning Electronic microscopy (SEM) (FIG. 6A) and transmission electron microscopy (TEM) (FIG. 6B) images of the particles. Right: magnified portion of the TEM image.

FIG. 7 shows time-dependent release of Rhodamine B dye from the photoexcited particles into water according to an embodiment of the current invention. The arrow indicates the time at which the azobenzene activation light was turned on.

FIGS. 8A-8C show confocal microscope images of the photocontrolled staining of the nuclei of PANC-1 cancer cells. Plasma membrane impermeable propidium iodide (PI) molecules were loaded in the pores of LAMS and the dye loaded particles were incubated with the cells for 3 hours in the dark. The cells were then exposed to the activation beam for 1 to 10 min. After further incubation in the dark for 10 min, the cells were examined with confocal microscopy (λex=337 nm) FIG. 8A. Cells incubated with the PI-loaded LAMS and illuminated for 0 (a), 1 (b), 3 (c), or 5 min (f) under a constant ˜0.2 W/cm², 413 nm light or with different light intensities (˜0.01 (d) or ˜0.1 W/cm² (e) for 5 min at a 413 nm light). FIG. 8B. PANC-1 cells incubated with the PI-loaded LAMS (g), free PI molecules (h), or empty LAMS (i) were kept in the dark and exposed to a 413 nm light. FIG. 8C. Cells incubated with the PI-loaded LAMS were illuminated with ˜0.2 W/cm², 676 nm light for 0 (j), 1 (k) or 5 min (l). Scale bar: 30 μm.

FIGS. 9A-9C show light-triggered delivery of the anticancer drug camptothecin (CPT) inside PANC-1 cancer cells to induce apoptosis according to an embodiment of the current invention. CPT molecules were loaded into the pores of the LAMS and a homogeneous suspension of the CPT-loaded particles (10 μg/ml) was added to the cells which were incubated in Lab-Tek chamber slides for 3 hrs in dark. The cells were then irradiated under ˜0.1 W/cm², 413 nm light for 1 to 10 min, again incubated in the dark for 48 hours, and double-stained with propidium iodide/Hoechst 33342 solution (1:1). FIG. 9A. CPT-loaded particles were incubated with cancer cells and illuminated for 1 (a), 3 (b), 5 (c) or 10 min (d, e, f). FIG. 9B. As controls, pure cells (no particles) were exposed to the light for 10 min (g), and cells including the CPT-unloaded LAMS were exposed for 5 (h) or 10 min (i). FIG. 9C. Untreated pure cells (j), cells incubated with CPT-unloaded (k) or -loaded (l) LAMS were kept in the dark for 48 hours. Scale bar: 30 μm.

FIG. 10 shows in vitro cytotoxicity assay. 5000 PANC-1 or SW480 cancer cells were incubated with different concentrations of CPT-loaded or unloaded particles in 96 well cell culture plates. After incubation for 72 hours following the light excitation, the numbers of surviving cells were counted using the cell counting kit. The viability is shown as the percentage of the viable cell number in treated wells compared to untreated wells. All experiments were performed in triplicate, and the results are shown as means±SD. LAMS: cells treated with the LAMS of 10 or 100 μg/ml. CPT: CPT was loaded (+) or absent in the LAMS. Light: cells were exposed to blue light (wavelength 413 nm) for 0, 1, 3, 5 or 10 min, followed by incubation for 72 hours.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited herein are incorporated by reference as if each had been individually incorporated.

The term “light” as used herein is intended to have a broad meaning to include electromagnetic radiation irrespective of wavelength. For example the term “light” can include, but is not limited to, infrared, visible, ultraviolet and other wavelength regions of the electromagnetic spectrum. In addition, the term “operable by light” is not limited to a single photon process, i.e., it may involve a single photon transfer, two photon transfer or multiple photon transfer.

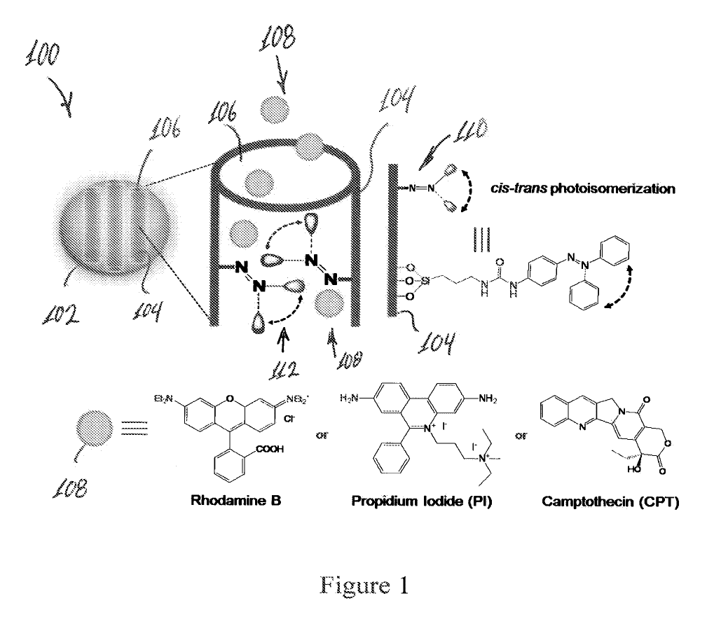

FIG. 1 is a schematic illustration of a nanodevice 100 according to an embodiment of the current invention. The nanodevice 100 has a containment vessel 102 defining a storage chamber 104 therein and defining at least one port 106 to provide transfer of molecules 108 into and/or out of the storage chamber 104. The nanodevice 100 also has an impeller 110 attached to the containment vessel 102. (The term “impeller” as used herein is intended to have a broad meaning to include structures which can be caused to move and which can in turn cause molecules located proximate the impeller to move in response to the motion of the impeller.) The impeller 110 is operable to impart motion to the molecules 108 to cause the molecules to at least one of enter into or exit from the storage chamber 104 of the containment vessel 102. The nanodevice 100 has a maximum dimension of less than about 400 nm and greater than about 50 nm. When the nanodevice 100 is greater than about 400 nm, it becomes too large to enter into biological cells. On the other hand, when the nanodevice 100 is less than about 50 nm, it becomes less able to contain a useful number of molecules therein. Furthermore, when the nanodevices are less than about 300 nm, they become more useful in some applications to biological systems. For some embodiments of the current invention, nanodevices having a maximum dimension in the range of about 50 nm to about 150 nm are suitable.

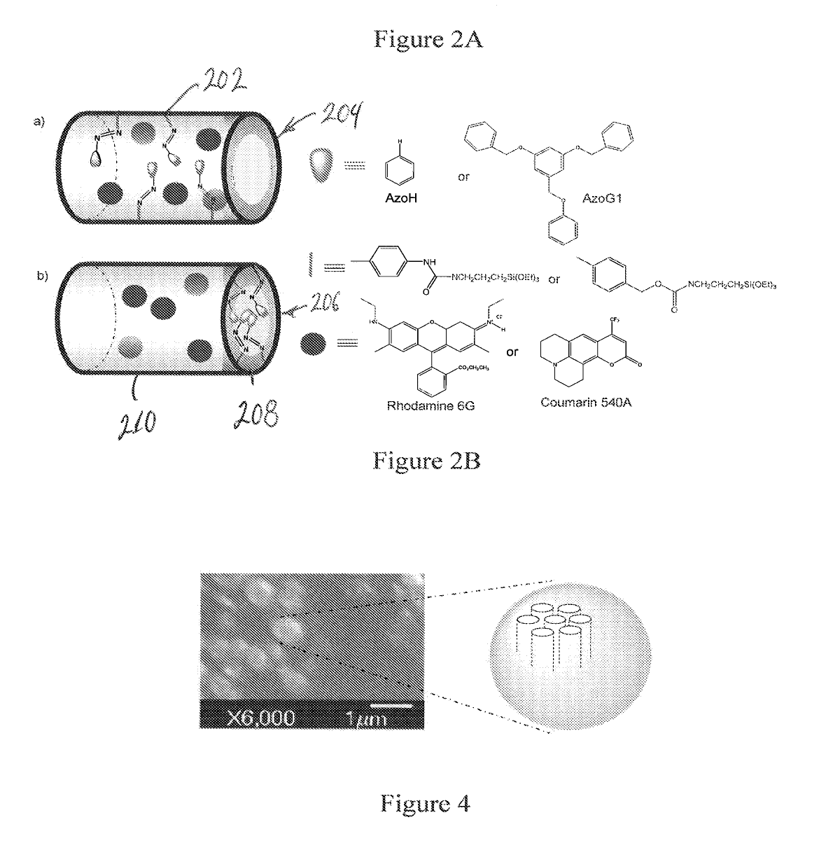

The nanodevice 100, according to some embodiments of the current invention, can have a plurality of impellers 112 attached to the containment vessel 102 in a number and arrangement so that they block molecules of interest (such as molecules 108) from entering and/or exiting from the storage chamber 104 of the containment vessel 102 while they are static, but can impel molecules of interest 108 to enter and/or exit the storage chamber 104 of the containment vessel 102 while they are in operation. FIG. 2A is a schematic illustration of a portion of a containment vessel showing a storage chamber 202 which can be, but is not limited to, one of a plurality of pores of a mesoporous silica nanoparticle. In this embodiment, the plurality of impellers 204 can be attached to the walls of the storage chamber 202. The particular molecules selected to be used as the impellers 204 are chosen taking into consideration the size of the storage chamber 202 and the size of the molecules that will be stored in the storage chamber 202. In operation, the impellers are driven by an energy transfer process. The energy transfer process can be, but is not limited to, absorption and/or emission of electromagnetic energy. For example, illuminating the nanodevice with light at an appropriate wavelength can cause the plurality of impeller to wag back and forth between two molecular shapes. The motion of the plurality of impellers 204 causes motion of molecules of interest into and/or out of the storage chamber 202. On the other hand, in the absence of excitation energy, the plurality of impellers can remain substantially static, at least for time periods long enough for the desired application, to act as impediments to block the molecules of interest from exiting and/or entering the storage chamber.

FIG. 2B is a schematic illustration of another embodiment of the current invention in which a plurality of impellers 206 are attached proximate a port 208 of storage chamber 210. The storage chamber 210 can be similar to or substantially the same as storage chambers 104 and 202. In this case the impellers 206 are selected to be of a size such that they cannot easily fit through the port 208 of the storage chamber 210. Furthermore, impellers 206 are selected to be of a size and are attached in a quantity and arrangement such that they impel molecules of interest into and/or out of the storage chamber 210 while the impellers are in motion, but block molecules of interest from exiting or entering the storage chamber 210 while they are static.

The containment vessels can be, but are not limited to, mesoporous silica nanoparticles according to some embodiments of the current invention. The impellers 112, 204 and 206 can be, but are not limited to, azobenzenes according to some embodiments of the current invention. For example, the azobenzenes can include the following:

1. One phenyl ring derivatized with a functional group that enables attachment to the silica support. The list of suitable functional groups contains but is not limited to: alcohols, (—ROH), anilinium amines (—NH₂) primary amines (—RNH₂), secondary amines (—R₁R₂NH), azides (N₃), alkynes (RC≡CH), isocyanates (—RNCO), isothiocyanates (—RNCS), acid halides (RCOX), alkyl halides (RX) and succinimidyl esters.

2. They can be derivatized with functional groups on the other phenyl ring (which is the moving end of the machine). The list of these functional groups includes but is not limited to: —H (here the phenyl ring is underivatized), esters (—OR), primary and secondary amines, alkyl group, polycyclic aromatics, and various generations of dendrimers. The bulkiness of these functional groups can be designed for specific systems. For example, large dendritic functionalities might be required when very large pore openings or very small guest molecules are employed.

Impellers Based on Redox of Copper Complexes

Impellers according to some embodiments of the current invention can include a group of copper complexes. The complexes can include bifunctional bidentate stators that contain diphosphine and/or diimine bidentate metal chelators on one end of the stator, while at the other end functionalities such as alkoxysilanes (for immobilization on silica and silicon substrates) and thiols (for immobilization on gold substrates) are present.

The copper complexes can contain a rotator that is a rigid bidentate diimine metal chelator, which rotates and changes the shape of the overall molecule upon redox or photons.

These copper complexes exist in two oxidation states, each of which corresponds to a specific shape. Copper (I) is tetrahedral while copper (II) is square planar.

The different oxidation states, and hence different shapes that are caused by a 90° rotation of the rotator, can be generated in three ways: Reduction and oxidation (1) using electrodes and an electric current (2) by use of chemical reducing and oxidizing agents, and (3) by the photo-excitation of light of the appropriate wavelength.

The molecules of interest to be stored in and released from the containment vessels can include, but are not limited to, biologically active substances. The term “biologically active substance” as used herein is intended to include all compositions of matter that can cause a desired effect on biological material or a biological system and may include in situ and in vivo biological materials and systems. The biologically active substance may be selected from such substances that have molecular sizes such that they can be loaded into the nanodevices, and can also be selected from such substances that don't react with the nanodevices. A biological system may include a person, animal or plant, for example.

Biologically active substances may include, but are not limited to, the following:

(1) Small molecule drugs for anticancer treatment such as camptothecin, paclitaxel and doxorubicin;

(2) Ophthalmic drugs such as flurbiprofen, levobbunolol and neomycin;

(3) Nucleic acid reagents such as siRNA and DNAzymes;

(4) Small molecule antioxidants such as n-acetylcysteine, sulfurophane, vitamin E, vitamin C, etc.;

(5) Small molecule drugs for immune suppression such as rapamycin, FK506, cyclosporine; and

(6) Any pharmacological compound that can fit into the nanodevice, e.g., analgesics, NSAIDS, steroids, hormones, anti-epileptics, anti-arrythmics, anti-hypentensives, antibiotics, antiviral agents, anticoagulants, platelet drugs, cardiostimulants, cholesterol lowering agents, etc.

Molecules of interest can also include imaging and/or tracking substances. Imaging and/or tracking substances may include, but are not limited to, dye molecules such as propidium iodide, fluorescein, rhodamine, green fluorescent protein and derivatives thereof.

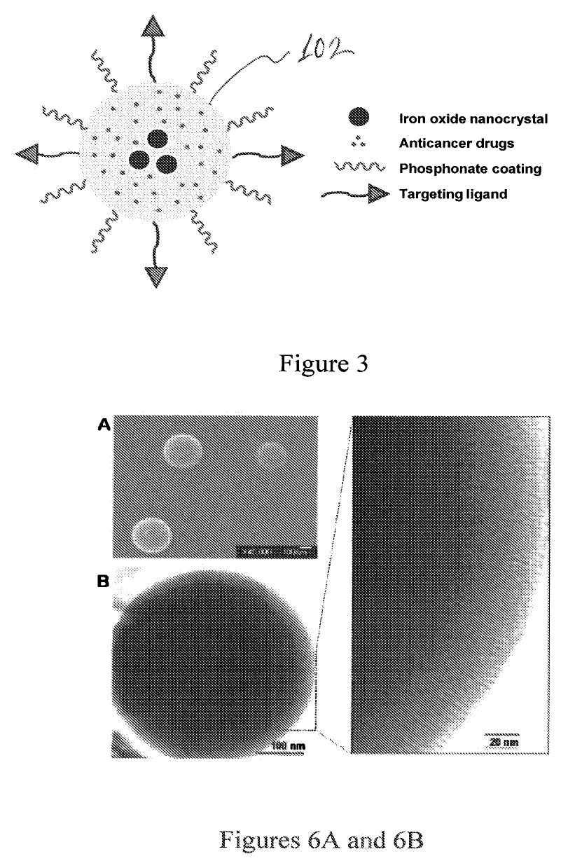

FIG. 3 is a schematic illustration to facilitate the explanation of additional embodiments of the current invention. For the sake of clarity, FIG. 3 does not show storage chambers, such as a plurality of pores of a mesoporous silica nanoparticle, and does not show impellers. However, it should be understood that they can be present in addition to the features illustrated in FIG. 3. According to some embodiments of the current invention, the nanodevices, such as nanodevice 100, can include a plurality of anionic molecules attached to the surface of the nanodevice as is illustrated schematically in FIG. 3. For example the anionic molecules can be phosphonate moieties attached to the outer surface of the nanodevice to effectively provide a phosphonate coating on the nanodevice. For example, the anionic molecules can be trihydroxysilylpropyl methylphosphonate molecules according to an embodiment of the current invention.

A phosphonate coating on the containment vessel, such as containment vessel 102, can provide an important role in some biological applications according to some embodiments of the current invention. This phosphonate coating can provide a negative zeta potential that is responsible for electrostatic repulsion to keep such submicron structures dispersed in an aqueous tissue culture medium, for example. This dispersion can also be important for keeping the particle size limited to a size scale that allows endocytic uptake (i.e., hinders clumping). In addition to size considerations, the negative zeta potential may play a role in the formation of a protein corona on the particle surface that can further assist cellular uptake in some applications. It is possible that this could include molecules such as albumin, transferrin or other serum proteins that could participate in receptor-mediated uptake. In addition to the role of the phosphonate coating for drug delivery, it can also provide beneficial effects for molecule loading according to some embodiments of the current invention. (See co-pending application number PCT/US08/13476, co-owned by the assignee of the current application, the entire contents of which are incorporated by reference herein.)

The nanodevice 100 can also be functionalized with molecules in additional to anionic molecules according to some embodiments of the current invention. For example, a plurality of folate ligands can be attached to the outer surface of the containment vessel 102 according to some embodiments of the current invention, as is illustrated schematically in FIG. 3 (impellers not shown for clarity).

In some embodiments of the current invention, the nanodevice 100 can also include fluorescent molecules contained in or attached to the containment vessel 102. For example, fluorescent molecules may be attached inside the pores of mesoporous silica nanoparticles according to some embodiments of the current invention. For example, the fluorescent molecules can be an amine-reactive fluorescent dye attached by being conjugated with an amine-functionalized silane according to some embodiments of the current invention. Examples of some fluorescent molecules, without limitation, can include fluorescein isothiocyanate, NHS-fluorescein, rhodamine B isothiocyanate, tetramethylrhodamine B isothiocyanate, and/or Cy5.5 NHS ester.

In further embodiments of the current invention, the nanodevices 100 may further comprise one or more nanoparticle of magnetic material formed within the containment vessel 102, as is illustrated schematically in FIG. 3 for one particular embodiment. For example, the nanoparticles of magnetic material can be iron oxide nanoparticles according to an embodiment of the current invention. However, the broad concepts of the current invention are not limited to only iron oxide materials for the magnetic nanoparticles. Such nanoparticles of magnetic material incorporated in the submicron structures can permit them to be tracked by magnetic resonance imaging (MRI) systems and/or manipulated magnetically, for example.

In further embodiments of the current invention, the nanodevices 100 may further comprise one or more nanoparticle of a material that is optically dense to x-rays. For example, gold nanoparticles may be formed within the containment vessel 102 of the nanodevice 100 according to some embodiments of the current invention.

### Example 1

In the following example according to an embodiment of the current invention, we show that continuous excitation at 457 nm, a wavelength where both the cis and trans conformers absorb, produces constant isomerization reactions that cause continual dynamic wagging of the untethered terminus and impel molecules through the pores. In addition, we show that the dynamic control of transport can be made to occur in 400 nm diameter particles containing 2 nm diameter pores in the current example.

In this example, we demonstrate that the dynamic motion of azobenzene derivatives can be used to control the transport of molecules trapped in the mesopores of silica nanoparticles. We report the use of azobenzene derivatives as both impellers and gatekeepers in and on mesoporous silica nanoparticles, such that guest molecules are expelled from the particles under photocontrol. We designed spherical particles with diameters of about 400 nm, a small azobenzene derivative, AzoH (FIG. 2A), to attach to the pore interiors, and a larger azobenzene derivatized with a G1 Frechet dendron (AzoG1) to attach to the pore openings (FIG. 2B). Our prior photophysical studies have shown that switching of immobilized azobenzenes occurs inside of mesopores; the trans to cis isomerization quantum yield at 450 nm is 0.36 and that for cis to trans is 0.64 (Sierocki, P. M., H.; Dragut, P.; Richardt, G.; Vogtle, F.; De Cola, L.; Brouwer, F. A. M.; Zink, J. I. J. Phys. Chem. B 2006, 110, 24390-24398). Continuous excitation at this wavelength produces constant isomerization reactions and results in continual dynamic wagging of the untethered terminus. In the experiments reported here, azobenzene-modified pores are loaded with luminescent probe molecules, azobenzene motion is stimulated by light, and luminescence spectroscopy is used to monitor the photoinduced expulsion of the probe from the particles that is caused by the azobenzene motion. The relative efficiency of expulsion of the small probe molecules during radiation to retention in the dark is dependent on the position of the azobenzene in the pore, the concentration, and the size of the azobenzene moving part.

The solid supports for the azobenzene machines (nanodevices in this embodiment) are ˜400 nm diameter particles that contain ordered 2D hexagonal arrays of tubular pores (4 nm lattice spacing) prepared by a base catalyzed sol-gel method (Kresge, C. T.; Leonowicz, M. E.; Roth, W. J.; Vartuli, J. C.; Beck, J. S. Nature 1992, 359, 710-712; Huh, S.; Wiench, J. W.; Yoo, J. C.; Pruski, M.; Lin, V. S. Y. Chem. Mater. 2003, 15, 4247-4256). The pores are templated by cetyltrimethylammonium bromide (CTAB) surfactants, and tetraethylorthosilicate (TEOS) is used as the silica precursor. Empty pores are obtained by template removal using solvent extraction or calcination. The ordered structure of the mesopores is confirmed by X-ray diffraction and the particle morphology by scanning electron microscopy (FIG. 4).

Two synthetic approaches were chosen to derivatize the silica in the desired region. To evenly derivatize the interiors of the mesopores, azobenzene was first coupled to the linker molecule isocyanatopropyltriethoxysilane (ICPES), and the machine-linker species was then added to the sol during particle synthesis and allowed to co-condense into the silica framework. The template was removed by solvent extraction. This synthetic approach will be termed the co-condensation method (CCM). To attach the AzoG1 primarily at the pore openings, the calcined mesostructured particles were treated with ICPES followed by coupling. The large azobenzenes cannot penetrate deep inside the pores and the first to react block access to the rest. This approach will be termed the post-synthesis modification method (PSMM). For all the syntheses, reagents were purchased from Aldrich and used as received with the exception of PhMe and ICPES, which were purified by distillation. The synthesis of AzoG1 has been previously reported (Sierocki, P. M., H.; Dragut, P.; Richardt, G.; Vogtle, F.; De Cola, L.; Brouwer, F. A. M.; Zink, J. I. J. Phys. Chem. B 2006, 110, 24390-24398), the entire contents of which are hereby incorporated by reference.

Preparation of AzoH-modified materials via the CCM.

The synthesis of AzoH-modified materials is derived from a previously reported synthetic methodology (Liu, N.; Dunphy, D. R.; Rodriguez, M. A.; Singer, S.; Brinker, C. J., Chem. Comm. 2003, 10, 1144-1145). 4-phenylazoaniline was first reacted with ICPES to form a carbamide linkage by refluxing 0.2840 g of the azo with 1.42 mL of ICPES in 10 mL of EtOH under N₂ for 4 h. During the coupling reaction, a surfactant solution (Huh, S.; Wiench, J. W.; Yoo, J. C.; Pruski, M.; Lin, V. S. Y. Chem. Mater. 2003, 15, 4247-4256) was prepared in the other flask: 2.0 g of CTAB, 7.0 ml, of 2M NaOH, and 480 g of the deionized H₂O were mixed and stirred for 30 minutes at 80° C. To this solution, 9.34 g of the tetraethylorthosilicate (TEOS) and the coupled AzoH-ICPES machine were slowly added with vigorous stirring. After 2 h of stirring at 80° C., the particles were filtered and thoroughly washed with MeOH and deionized H₂O. Template removal was accomplished by suspending 1 g of the as-synthesized particles in 100 mL of MeOH with 1 mL of concentrated HCl and heating at 60° C. for 6 h.

Preparation of AzoG1-modified materials via the PSMM.

Pure mesoporous silica nanoparticles were prepared according to published literature procedure (Huh, S.; Wiench, J, W.; Yoo, J. C.; Pruski, M.; Lin, V. S. Y. Chem. Mater. 2003, 15, 4247-4256). The CTAB surfactant was removed by calcination at 550° C. for 5 hours. Attachment of the ICPES linker was accomplished by suspending 100 mg of the calcined particles in 10 mL of a 10 mM solution of ICPES in dry PhMe and refluxing for 12 h under N₂. ICPES-modified particles were filtered and thoroughly washed with PhMe and then placed in a 1 mM solution of AzoG1 in PhMe and refluxed for 12 h under N₂. The AzoG1-modified particles were recovered by filtration, washed thoroughly with PhMe, and then dried under vacuum.

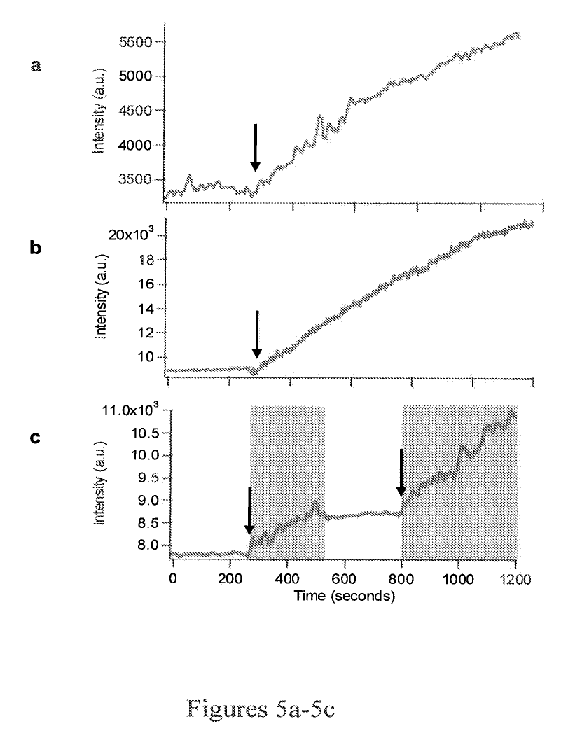

In order to use the azobenzene motion as an impeller, the small AzoH was attached onto the pore interiors using the CCM. Real time measurements of the rate of expulsion of two different dyes, Coumarin 540A and Rhodamine 6G, were made. The pores were loaded with dye molecules by soaking the particles in 1 mM solutions of the dye overnight and then washed to remove adsorbed molecules from the surface. 15 mg of dye-loaded particles were placed in the bottom of a cuvette and 12 mL of MeOH was carefully added. A 1 mW, 457 nm probe beam directed into the liquid was used to excite dissolved dye molecules that are released from the particles. The spectrum was recorded as a function of time at 1 sec intervals. After 5 minutes, a 9 mW, 457 nm excitation beam was used to directly irradiate the functionalized particles and excite the azobenzenes' motion. Plots of the dissolved dye luminescence intensity at the emission maximum as functions of time (the release profiles) indicate that the particles hold the guest molecules but expel them when stimulated (FIG. 5a). As a control experiment to verify that azobenzene excitation drives the release, the particles were irradiated with equal power at a wavelength (647 nm) at which the azobenzene does not absorb. The red light had no effect on the release. These results demonstrate that the system only responds to wavelengths that drive the large amplitude azobenzene motion.

The expulsion of molecules from pores containing azobenzene molecules attached internally probably involves an “impeller” mechanism. However, the broad concepts of the current invention are not limited to this specific mechanical visualization of a possible mechanism. Prior to excitation, dye molecules are held inside the particles because the pores are considerably congested by the static azobenzene machines and a facile pathway for escape is not available. Excitation of the azobenzenes causes them to wag back and forth, effectively imparting motion to the trapped dye molecules and allowing them to traverse the pore interior until they escape. The concentration at which azobenzene machines are tethered to the pore interiors determines the amount of congestion inside the mesopores, and therefore affects the ability to trap dye molecules in the dark. The effective concentration of the AzoH machines tethered inside the mesopores can be varied by changing the amount of the AzoH-ICPES precursor that is added to the TEOS sol during particle synthesis. When particles are prepared such that the concentration of azobenzene molecules doped into the pores is decreased by a factor of three, very slow diffusion of the dye molecules through the pores occurs in the dark and the system is leaky. It is likely that the decreased amount of azobenzene creates enough free space inside the mesopore such that the dye molecules can diffuse around in the dark, and are never completely trapped.

A second method of exploiting dynamic motion is to attach larger azobenzene derivatives at the pore orifices such that the machines can gate the pore openings in the dark. Static large molecules clog the entrances, but dynamic movement can provide intermittent openings for small molecules to slip through. In this gatekeeping approach, the size of the machine selected is an important factor affecting nanovalve operation according to this embodiment of the current invention. The azobenzene derivative must be sufficiently large such that it can block the nanopore entrances when it is static, and mobile enough when irradiated to provide openings through which molecules can escape. AzoG1 was selected because its 1 nm size suggested that several would be sufficient to block the 2 nm pores. Minimal leakage of probe molecules is observed prior to excitation but irradiation allows rapid escape (FIG. 5b). The smaller derivative AzoH does not sufficiently block the openings and leakage is observed when the molecules are static.

The fact that the dynamic motion responsible for controlling molecular transport can be photoresponsively turned on and off enables the systems to be externally regulated such that the expulsion of dye molecules from the mesopores can be started and stopped at will. The release profile of Coumarin 540A from AzoG1-treated particles where the excitation is sequentially turned on and off is shown in FIG. 5c. The pore openings are adequately blocked in the dark and dyes are expelled from the particles only upon excitation of the AzoG1. Remote control of the flow of molecules out of mesopores is thus demonstrated.

The functional nanoparticles described in this example utilize the photo-controllable static and dynamic properties of azobenzene derivatives in and on mesopores. Luminescent probe molecules enable the function to be sensitively monitored. This helps explain the usefulness of nanodevices according to some embodiments of the current invention for selectively trapping and releasing molecules such as drugs on demand.

### Example 2

Mesoporous silica nanoparticles with an average diameter of about 200 nm can enter cells and have been used as gene transfection reagents, cell markers, and carriers of molecules such as drugs and proteins (C. Y. Lai, B. G. Trewyn, D. M. Jeftinija, K. Jeftinija, S. Xu, S. Jeftinija, V. S. Y. Lin, J. Am. Chem. Soc., 2003, 125, 4451; Y. S. Lin, C. P. Tsai, H. Y. Huang, C. T. Kuo, Y. Hung, D. M. Huang, Y. C. Chen, C. Y. Mou, Chem. Mater., 2005, 17, 4570; D. R. Radu, C. Y. Lai, K. Jeftinija, E. W. Rowe, S. Jeftinija, V. S. Y. Lin, J. Am. Chem. Soc., 2004, 126, 13216; Slowing, II, B. G. Trewyn, V. S. Lin, J Am Chem Soc, 2007, 129, 8845; M. Arruebo, M. Galan, N. Navascues, C. Tellez, C. Marquina, M. R. Ibarra, J. Santamaria, Chem. Mater., 2006, 18, 1911; K. Weh, M. Noack, K. Hoffmann, K. P. Schroder, J. Caro, Microporous Mesoporous Mater., 2002, 54, 15; E. Besson, A. Mehdi, D. A. Lerner, C. Reye, R. J. P. Corriu, J. Mater. Chem., 2005, 15, 803; J. Lu, M. Liong, J. I. Zink, F. Tamanoi, Small, 2007, 3, 1341).

In the following example according to an embodiment of the current invention, we describe the use of nanoimpeller-controlled mesostructured silica nanoparticles to deliver and release anticancer drugs into living cells upon external command. By using light-activated mesostructured silica (LAMS) nanoparticles, luminescent dyes and anticancer drugs are only released inside of cancer cells that are illuminated at the specific wavelengths that activate the impellers. The quantity of molecules released is governed by the light intensity and the irradiation time. Human cancer cells (a pancreatic cancer cell line, PANC-1 and a colon cancer cell line, SW 480) were exposed to suspensions of the particles and the particles were taken up by the cells. Confocal microscopy imaging of cells containing the particles loaded with the membrane-impermeable dye, propidium iodide (PI), shows that the PI is released from the particles only when the impellers are photoexcited (˜0.1 W/cm²), resulting in staining of the nuclei. The anticancer drug camptothecin (CPT) was also loaded into and released from the particles inside the cells under light excitation, and apoptosis was induced. Intracellular release of molecules is sensitively controlled by the light intensity, irradiation time, and wavelength, and the anticancer drug delivery inside of cells is regulated under external control.

The LAMS functionalized with azobenzene molecules were synthesized using modifications of reported processes (N. Liu, Z. Chen, D. R. Dunphy, Y. B. Jiang, R. A. Assink, C. J. Brinker, Angew. Chem. Int. Ed. Engl., 2003, 42, 1731; S. Angelos, E. Choi, F. Vogtle, L. DeCola, J. I. Zink, J. Phys. Chem. C, 2007, 111, 6589). In the resulting particles, azobenzene moieties were positioned in the pore interiors with one end attached to the pore walls and the other end free to undergo photoisomerization (FIGS. 1 and 2A). The morphology of the spherical particles with ordered arrays of the pores was proven by scanning electron microscopy (SEM) and transmission electron microscopy (TEM) (FIGS. 6A and 6B). The X-ray diffraction pattern exhibited a strong Bragg peak indexed as {100} at 2θ=2.43°, corresponding to a d-spacing of ˜3.6 nm. Analysis of the nitrogen sorption isotherm of the taken at 77 K indicated the BJH average pore diameter of 1.9±0.1 nm, BET surface area of 621.19 m² g⁻¹, and total pore volume of 0.248 cm³ g⁻¹. It was calculated from UV/Vis spectroscopy that the silica particles contain about 2.4 wt % of the azobenzene derivatives.

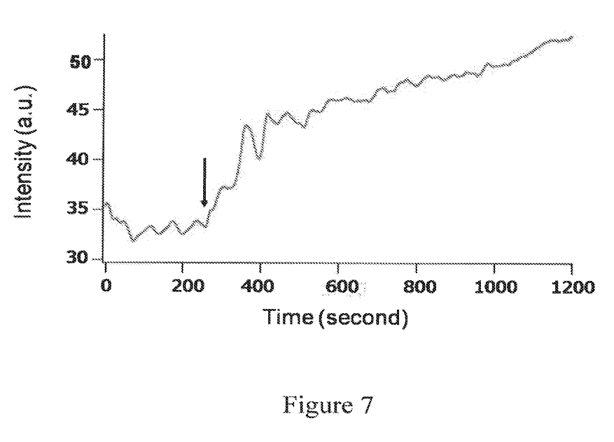

Controlled expulsion of the pore contents into solution was monitored by luminescence spectroscopy (S. Angelos, E. Choi, F. Vogtle, L. DeCola, J. I. Zink, J. Phys. Chem. C, 2007, 111, 6589). Hydrophilic Rhodamine B was chosen as a probe dye to verify that the moving parts are able to trap and release the probe molecules in an aqueous environment. The fluorescence emission spectrum of the Rhodamine B probe molecules that were released from the particles into water was recorded at one second intervals. The intensities at the emission maximum (λ˜575 nm) as a function of time are plotted in FIG. 7. The impellers in nanopores trap the probe molecules in the dark and promptly release them in response to the light excitation.

Based on the successful operation of the impeller in water, in vitro studies were carried out on two human cancer cell lines (PANC-1 and SW480). To detect the photoresponsive behavior of the impellers inside of cells, a membrane-impermeable dye, PI was chosen as the fluorescent probe molecule and loaded into the particles following the same procedure as that used for the Rhodamine B loading. The cells were cultured overnight on a Lab-Tek chamber slide system (Nalge Nunc International). After 3 h of incubation in the dark with a 10 μg/mL homogeneous suspension of PI-loaded LAMS containing ˜0.24 μg of the azobenzene machines, the cells were irradiated at 413 nm, a wavelength at which both cis and trans azobebenzene isomers have almost the same extinction coefficient. The cells were exposed to three different excitation fluences (˜0.01, 0.1, 0.2 W/cm²) with exposure times ranging from 0 to 5 min. As a control, the cells were also exposed to 676 nm, a wavelength at which azobenzene does not absorb, at the same light intensities for the same amounts of time as in the release experiments. After irradiation, the cells were again incubated in the dark for 10 min to allow the released PI to stain the nuclei of the cells, and then examined by confocal microscopy (λₑₓ=337 nm; Carl Zeiss LSM 310 Laser Scanning Confocal microscope).

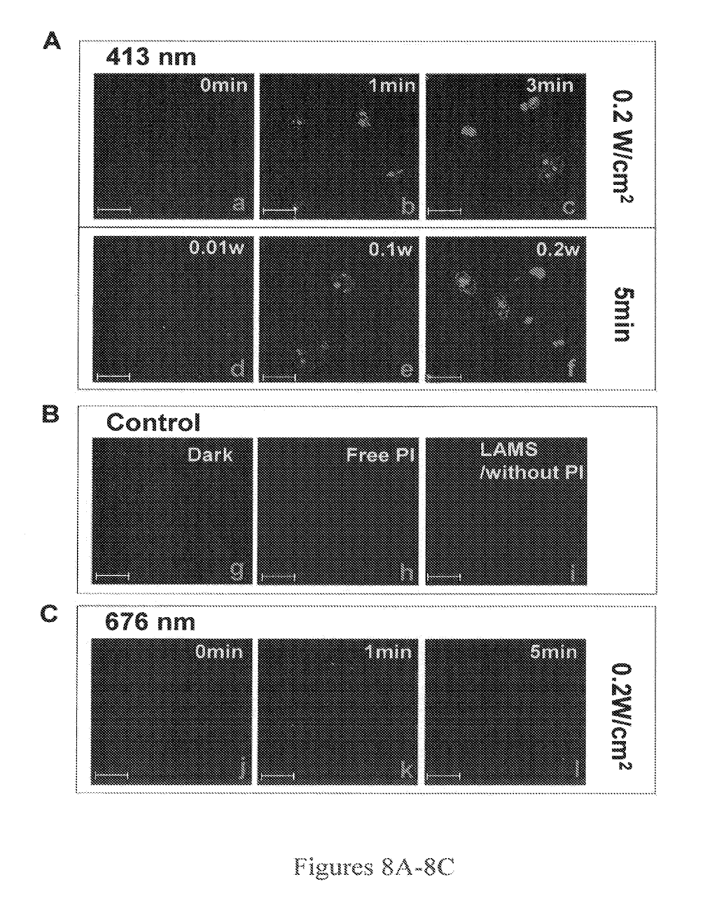

Confocal fluorescence images of the PANC-1 cells showed that only after the photo-activation of the azobenzene impellers was the PI released from the LAMS, resulting in staining of the cell nuclei (FIGS. 8A-8C). When the cells were irradiated for 5 min with 413 nm light of ˜0.2 W/cm² beam intensity, the nuclei were dyed red, but negligible dying of the nuclei was observed in the cells kept in the dark. For cells excited with a decreased intensity, ˜0.1 W/cm², the nuclei were stained to a lighter red, and no staining was observed from ˜0.01 W/cm² irradiation, which did not activate the impellers enough to enable them release much PI (FIG. 8A (d-f)). When exposed to different excitation times of up to 5 min under constant fluence of ˜0.2 W/cm² at 413 nm, the nuclei were stained increasingly redder with increasing activation time (FIG. 8A (a-c, f)), verifying that the amount of PI released is directly related to the total number of photons absorbed. The cells were not stained when the LAMS were irradiated at 676 nm (˜0.2 W/cm²) because that wavelength is not absorbed by the impellers (FIG. 8C). These results prove that the impeller operation can be regulated by the light intensity, excitation time, and specific wavelength, and that these controllable factors directly affect the amount of the pores' contents that is released. When cells were incubated with free PI that were not loaded into the particles, cell staining did not occur (FIG. 8B (h)), proving that the free PI molecules cannot enter the cells. The staining of the nuclei is thus caused only by the PI that is carried into the cells by the LAMS and released from the particles when they are photoexcited.

Similar results were obtained in experiments using colon cancer cells SW480. Staining of the nuclei was caused by illuminating the LAMS with ˜0.2 W/cm², 413 nm light. The LAMS particles function controllably in multiple cell types.

To test the ability of the LAMS to transport and then controllably release drug molecules inside cancer cells, the particles were loaded with the anticancer drug camptothecin (CPT). A 10 μg/mL homogeneous suspension of the drug-loaded particles was added to the cancer cells. After 3 hours of incubation in the dark, the cells were irradiated with ˜0.1 W/cm², 413 nm light for various excitation times (0 to 10 min). The power density of ˜0.1 W/cm² was chosen for this experiment based on the PI cell staining results. For the confocal cell imaging measurements, the irradiated cells were again incubated for 48 h in the dark and then stained with a 1:1 mixture solution of PI and Hoechst 33342 dye to investigate the cell death. As control experiments, cells incubated with empty LAMS particles and cells without any treatment were exposed to the excitation light.

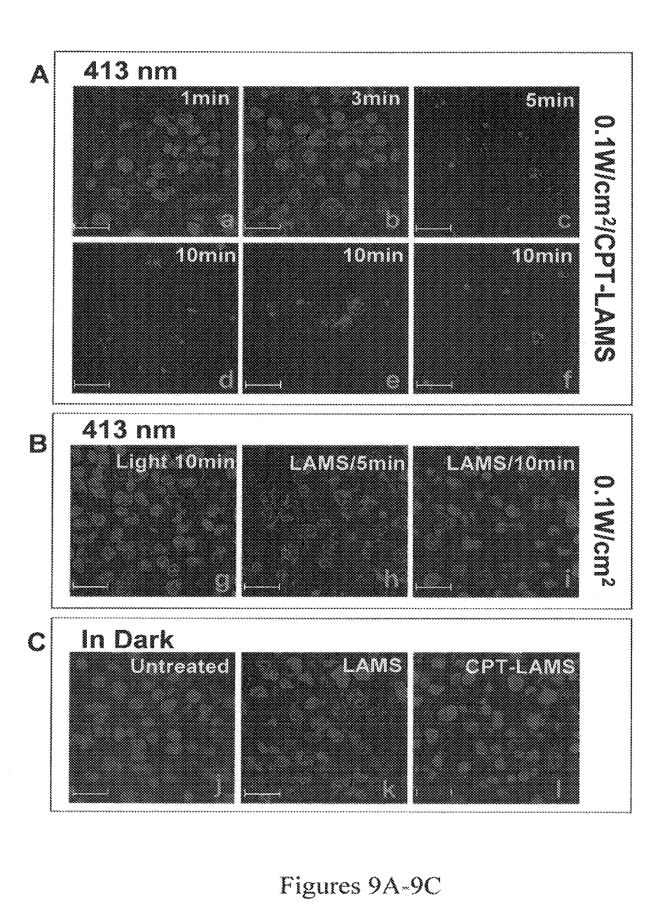

Cell death was induced under photocontrol. In the absence of light excitation, the CPT remained in the particles and the cells were not damaged (FIG. 9C (l)). Illumination, however, promptly expelled the CPT from the particles, causing cancer cell apoptosis that is demonstrated by nuclear fragmentation and chromatin condensation (J. Hasegawa, S. Kamada, W. Kamiike, S. Shimizu, T. Imazu, H. Matsuda, Y. Tsujimoto, Cancer Res, 1996, 56, 1713; F. Belloc, P. Dumain, M. R. Boisseau, C. Jalloustre, F. Reiffers, P. Bernard, F. Lacombe, Cytometry, 1994, 17, 59; Z. Darzynkiewicz, G. Juan. X. Li, W. Gorczyca, T. Murakami, F. Traganos, Cytometry, 1997, 27, 1) (FIG. 9A). The cell nuclei all fluoresced blue from the Hoechst 33342 dye while no red fluorescent cell nuclei stained by the PI dye were detected, confirming that the cell death did not result from cellular membrane damage but from apoptosis by the released CPT inside of the cells. The cells containing empty LAMS particles (no CPT) that were exposed to the excitation beam for 10 min did not undergo cell death, indicating that the LAMS particles are biocompatible with the living cells (FIG. 9B (h, i)). The ˜0.1 W/cm², 413 nm activation light beam did not affect the cell survival (FIG. 9B (g)). CPT suspended in PBS was not taken up by the cells due to its insolubility and thus did not kill the cells. These observations demonstrate that cancer cell apoptosis is caused only by the CPT released from the LAMS particles inside cells under external photocontrol.

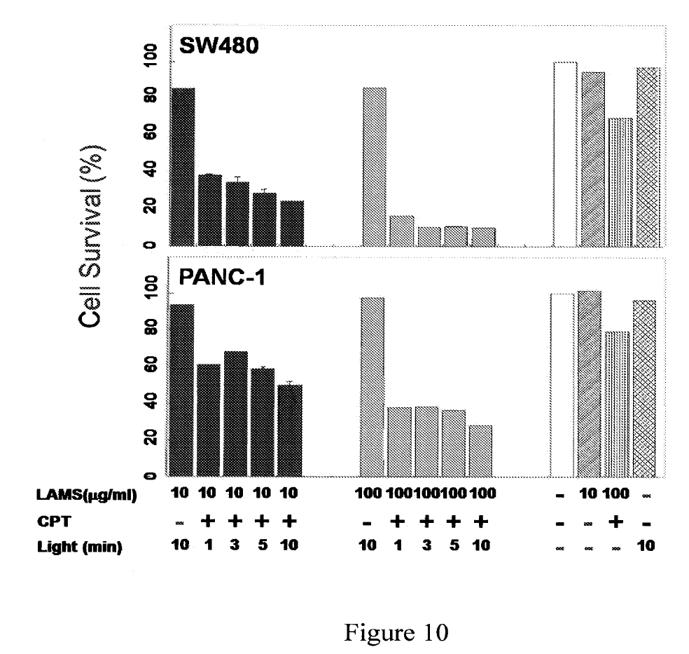

To further confirm that cell death was caused by the cytotoxicity of the CPT expelled from the particles, quantitative measurements of cell viability were made for another set of the same samples (10 μg/mL particles incubated with cells) placed in 96-well plates. After incubation with LAMS with and without CPT loaded and illumination with ˜0.1 W/cm², 413 nm, the cells were kept in the incubator for an additional 72 hours. The number of surviving cells was then counted using a cell counting kit from Dojindo Molecular Technologies, Inc. (J. Lu, M. Liong, J. I. Zink, F. Tamanoi, Small, 2007, 3, 1341). The result showed that the cell death induced by CPT only occurred under light illumination, and the cell death rate increases with longer cell illumination time, which is consistent with the cell morphologic observations. The surviving cells decreased to about half after 10 min of photoexcitation of the impellers (FIG. 10). At a higher concentration (100 μg/mL) of the particles, cell survival decreased more dramatically; only ˜40% of the PANC-1 cells and ˜14% of the SW480 survived the released CPT after 10 min of light excitation (FIG. 10).

In summary, we demonstrated that the biocompatible nanoimpeller-based delivery system regulates the release of molecules from the nanoparticles inside of living cells. This nanoimpeller system may open a new avenue for drug or other guest molecule delivery under external control at a specific time and location for photo-therapy. Manipulation of the machine is achieved by remote control by varying both the intensity of the light and time that the particles are irradiated at the specific wavelengths where the azobenzene impellers absorb. The CPT loading (˜0.6 wt %) in the LAMS was higher than that for underivatized mesostructured silica (˜0.06 wt %) (J. Lu, M. Liong, J. I. Zink, F. Tamanoi, Small, 2007, 3, 1341), possibly because of the hydrophobic molecular interactions between azobenzene moieties and CPT. When excited at 413 nm, the azobenzenes' continuous photoisomerization acts as an impeller and expels CPT out of the pores. The light intensity needed to activate the impellers, ˜0.1 W/cm² at 413 nm, does not damage the cells. The action of the LAMS is monitored by release of PI and the consequent staining of the cell nuclei, and by the release of CPT that induces apoptosis. The delivery and release capability of light-activated mesostructured silica particles containing molecular impellers can provide a novel platform for nanotherapeutics with both spatial and temporal external control according to some embodiments of the current invention.

### Experimental Procedures

Synthesis of Light-Activated Mesostructured Silica Nanoparticles:

The chemicals for the particle synthesis were purchased from Sigma-Aldrich. The bifunctional modification strategy (P. N. Minoofar, B. S. Dunn, J. I. Zink, J. Am. Chem. Soc., 2005, 127, 2656; P. N. Minoofar, R. Hernandez, S. Chia, B. Dunn, J. I. Zink, A. C. Franville, J Am Chem Soc, 2002, 124, 14388) was used to incorporate 4-phenylazoaniline (4-PAA) into the interiors of the particle pores. Organosilane molecules containing azobenzene moieties were first generated via coupling reaction of 0.142 g of the 4-PAA with 0.71 mL of the isocyanatopropylethoxysilane (ICPES) linker in 5 mL ethanol under N₂ for 4 hours. In another flask, 1 g of the templating agent dodecyltrimethylammonium bromide (DTAB), 3.5 mL of 2M NaOH, and 480 g of deionized H₂O were stirred for 30 min at 80° C. To this surfactant solution, 4.67 g of the tetraethylorthosilicate (TEOS) and the ethanol solution containing the azobenzene machines were slowly added and vigorously stirred. After 2 h the particles were filtered and washed with MeOH. The surfactant was extracted by stirring 1 g of the particles in 100 mL of MeOH with 1 mL of concentrated HCl solution for 6 h at 60° C.

Dye Loading Procedure:

The probe molecules, Rhodamine B or propidium iodide, are loaded into the mesopores by soaking and stirring ˜20 mg of the particles in a 1 mM aqueous solution of the dye at room temperature for 12 h. The suspensions of particles in aqueous dye solution were then centrifuged for ˜10 min, and the supernatant was decanted. The particles were suspended again in deionized water and sonicated for at least 10 min. This step was repeated at least twice to thoroughly remove the dyes adsorbed onto the particle surface. The particles were then dried at room temperature.

Anticancer Drug Loading Procedure:

A solution of 0.6 mL dimethylsulfoxide (DMSO) containing 1 mg of the CPT molecules was prepared, and 10 mg of the LAMS was added. After stirring the suspension for 24 h, the mixture was centrifuged for 10 min and the supernatant solution removed. The CPT-loaded LAMS were then dried under vacuum. To determine the amount of CPT molecules loaded in the LAMS, the drug-loaded LAMS were dissolved and sonicated with 4 mL DMSO, placed in a quartz cuvette as in the release experiment, and irradiated by ˜0.2 W/cm², 413 nm light for 10 min. The DMSO suspension of the particles was then centrifuged and the UVNis absorption spectrum of supernatant solution containing the released CPT molecules was measured. The concentration of CPT calculated from the absorbance was ˜0.09 mM. To confirm that most of the loaded CPT molecules were released from the particles, the supernatant taken out for the absorbance measurement was placed back into the cuvette with the centrifuged particles, excited for 50 min, and the absorbance measurement was repeated. It was determined that about 0.12 mg of CPT molecules was loaded into 20 mg of the particles.

Spectroscopic Setup for Controlled Release Experiments:

The Rhodamine B-loaded particles were carefully placed on the bottom of a cuvette filled with deionized H₂O. The liquid above powder was monitored continuously by a 10 mW, 530 nm probe beam. The LAMS powder was activated with a 10 mW, 457 nm excitation beam. Both the cis and trans azobenzene isomers absorb at that wavelength with a conversion quantum yield of about 0.4 for trans to cis and 0.6 for cis to trans (P. Sierocki, H. Maas, P. Dragut, G. Richardt, F. Vogtle, L. D. Cola, F. A. Brouwer, J. I. Zink, J. Phys. Chem. B, 2006, 110, 24390). The release profiles are obtained by plotting the luminescence intensity at the emission maximum as a function of time.

Cell Culture:

PANC-1 and SW480 Cells were obtained from the American Type Culture Collection and were maintained in Dulbecco's modified Eagle's medium (DMEM) (GIBCO) and Leibovitz's L-15 medium (Cellgro) respectively, supplemented with 10% fetal calf serum (Sigma, MO), 2% L-glutamine, 1% penicillin, and 1% streptomycin stock solutions with regular passage.

Cell Death Assay:

Cell death was also examined by using the propidium iodide and Hoechst 33342 double-staining method. The cells incubated on a Lab-Tek chamber slide system were stained with propidium iodide/Hoechst 33342 (1:1) for 5 min after treatment with CPT-loaded LAMS or free LAMS followed by light irradiation, and then examined with fluorescence microscopy. The cell survival assay was performed by using the cell-counting kit from Dojindo Molecular Technologies, Inc. Cancer cells were seeded in 96-well plates (5000 cells/well) and incubated in fresh culture medium at 37° C. in a 5% CO₂/95% air atmosphere for 24 h. After incubation with LAMS with and without CPT loaded and illumination with ˜0.1 W/cm², 413 nm light, the cells were kept in the incubator for an additional 72 hours. The cells were then washed with PBS and incubated in DMEM with 10% WST-8 solution for another 2 h. The absorbance of each well was measured at 450 nm with a plate reader. Since the absorbance is proportional to the number of viable cells in the medium, the viable cell number was determined by using a previously prepared calibration curve (Dojindo Co.).

Statistical Analysis:

All results are expressed as mean values the standard deviation (SD). Statistical comparisons were made by using Student's t-test after analysis of variance. The results were considered to be significantly different at a P value <0.05.

In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1: A nanodevice, comprising:
a containment vessel defining a storage chamber therein and defining at least one port to provide transfer of molecules to or from said storage chamber; and
an impeller attached to said containment vessel,
wherein said impeller is operable for at least one of loading, unloading, or containing molecules within said containment vessel, and
wherein said nanodevice has a maximum dimension of less than about 400 nm and greater than about 50 nm.

2: The nanodevice of claim 1, wherein said nanodevice has a maximum dimension of less than about 300 nm and greater than about 50 nm, or less than about 150 nm and greater than about 50 nm.

3. (canceled)

4: The nanodevice of claim 1, wherein said nanodevice is operable in an aqueous environment.

5: The nanodevice of claim 1, wherein said impeller is operable by light illuminated thereon.

6: The nanodevice of claim 5, wherein said impeller comprises a molecule that undergoes a change in shape upon absorption of light illuminated thereon.

7: The nanodevice of claim 1, wherein said nanodevice consists essentially of biocompatible materials in a composition thereof.

8. (canceled)

9: The nanodevice of claim 1, wherein said containment vessel is a mesoporous silica nanoparticle defining a plurality of substantially parallel pores therein, said storage chamber being one of said plurality of substantially parallel pores.

10: The nanodevice of claim 9, wherein said impeller is a molecule selected from the group of azobenzene molecules that is attached to said mesoporous silica nanoparticle.

11: The nanodevice of claim 1, further comprising a plurality of anionic or electrostatic molecules attached to an outer surface of said containment vessel, wherein said anionic or electrostatic molecules provide hydrophilicity or aqueous dispersability to said nanodevice and are suitable to provide repulsion between other similar nanodevices.

12: The nanodevice of claim 11, wherein said anionic molecules comprise a phosphonate moiety.

13: The nanodevice of claim 11, wherein said plurality of anionic molecules are trihydroxysilylpropyl methylphosphonate.

14: The nanodevice of claim 1, further comprising folate ligands attached to said containment vessel.

15: The nanodevice of claim 1, further comprising a nanoparticle of magnetic material or of gold formed within said containment vessel of said nanodevice.

16: The nanodevice of claim 15, wherein said nanoparticle of magnetic material is an iron oxide nanoparticle.

17. (canceled)

18: A nanodevice, comprising:
a containment vessel defining a storage chamber therein and defining at least one port to provide transfer of molecules to or from said storage chamber; and
a plurality of impellers attached to said containment vessel,
wherein said plurality of impellers are of a structure and are arranged to substantially block molecules from entering and exiting said storage chamber of said containment vessel when said impellers are static and are operable to cause or allow said molecules to at least one of enter into or exit from said storage chamber of said containment vessel.

19: A composition of matter, comprising:
a plurality of nanoparticles, each defining a storage chamber therein; and
a guest material contained within said storage chambers defined by said nanoparticles, said guest material being substantially chemically non-reactive with said nanoparticles,
wherein said plurality of nanoparticles are operable to cause said guest material contained within said storage chambers to be released upon a transfer of energy to said plurality of nanoparticles from a source of energy external to said plurality of nanoparticles, and
wherein each nanoparticle of said plurality of nanoparticles has a maximum dimension of less than about 400 nm and greater than about 50 nm.

20: The composition of matter of claim 19, wherein said transfer of energy is an illumination of said plurality of nanoparticles with light.

21: The composition of matter of claim 19, wherein said nanoparticles are operable in an aqueous environment.

22. (canceled)

23: The composition of matter of claim 19, wherein each nanoparticle of said plurality of nanoparticles comprises a mesoporous silica nanoparticle defining a plurality of substantially parallel pores therein, said storage chamber being one of said plurality of substantially parallel pores.

24: The composition of matter of claim 19, wherein each nanoparticle of said plurality of nanoparticles comprises a molecule selected from the group of azobenzene molecules that is attached to said mesoporous silica nanoparticle.

25: The composition of matter of claim 19, wherein each nanoparticle of said plurality of nanoparticles comprises a surface coating of a hydrophilic silane.

26: The composition of matter of claim 19, wherein each nanoparticle of said plurality of nanoparticles comprises folate ligands attached thereto.

27: A method of administering at least one of a biologically active substance, a therapeutic substance, a neutraceutical substance, a cosmetic substance or a diagnostic substance, comprising:
administering a composition to at least one of a person, an animal, a plant, or an organism, said composition comprising nanoparticles therein, wherein said nanoparticles contain said at least one of a biologically active substance or a diagnostic substance therein; and
illuminating said nanoparticles of said administered composition with light to cause said at least one of said substances to be released from said nanoparticles.
